(19) United States
(12) Patent Application Publication
(10) Pub. No.: US 2004/0259232 A1
(43) Pub. Date: Dec. 23, 2004

(54) Organism cell auto-handling apparatus and its handling method

(75) Inventors: Yeou-Pin Guu, Taichung City (TW); Jinn-Fa Wu, Taichung City (TW)

Correspondence Address: BACON & THOMAS, PLLC, 625 SLATERS LANE, FOURTH FLOOR, ALEXANDRIA, VA, 22314

(73) Assignee: INDUSTRIAL TECHNOLOGY RESEARCH INSTITUTE, HSINCHU HSIEN (TW)

(21) Appl. No.: 10/754,720

(22) Filed: Jan. 12, 2004

(30) Foreign Application Priority Data
Jun. 19, 2003 (TW) 92116722

(51) Int. Cl. C12M 3/00

(52) U.S. Cl. 435/285.1; 435/286.2

(57) ABSTRACT

An organism cell auto-handling apparatus includes a platform, an organelle observation microscope unit, a piping device and a system controller. An organism cell auto-handling method using the aforesaid apparatus includes the steps of putting an organism cell sample on the platform, then using the organelle observation microscope unit to position a specific organelle of said organism cell sample and produce a corresponding coordinate data to the system controller, and then processing an organelle draw/injection action to the specific organelle by a micro needle of the piping device that is controlled by the system controller.

### BACKGROUND OF THE INVENTION

1. Field of the Invention

The present invention relates generally to the draw/injection handling of organelles of organism cells and, more particularly, to an organism cell auto-handling method and an organism cell auto-handling apparatus for the method.

2. Description of the Related Art

In order to obtain effective analysis when studying hereditary diseases or immunological diseases, such as senile dementia, mucopo lysaecharidoses, etc., it is necessary to study on the organelles, such as mitochondrion, Golgi apparatus, endoplasmic reticulum, etc., of the cell. Therefore, cell organelle draw handling is important.

Conventionally, there are two cell organelle draw methods. The first cell organelle draw method is to split a mass of cells, and then uses a centrifuge to separate different compositions from the cytoplasm. This method cannot obtain a specific organelle of each individual cell. The second cell organelle draw method is to put the organism cell sample on a platform, and then use a high-power light microscope to search a specific organelle from the organism cell, and then use a micro needle to pick up the organelle of the cell. The speed of this manual operation is slow, and its chance of failure is high. Further, because it is not easy to directly view the internal organelles of the cell through a light microscope, the illumination of ultraviolet light is necessary. However, the radiation of ultraviolet light affects the characteristics of the internal organelles of the cell.

Therefore, it is desirable to provide an organism cell auto-handling apparatus that eliminates the aforesaid drawbacks.

### SUMMARY OF THE INVENTION

It is the primary objective of the present invention to provide an organism cell auto-handling apparatus, which has the function of automatically and accurately drawing/injecting a specific organelle of an organism cell.

It is another objective of the present invention to provide an organism cell auto-handling apparatus, which accelerates the speed of the draw/injection of a specific cell organelle, and increases the change of success.

It is still another objective of the present invention to provide an organism cell auto-handling apparatus, which does not affect the characteristics of the cell organelle during searching.

It is still another objective of the present invention to provide an organism cell auto-handling method using the apparatus provided by the present invention.

According to one aspect of the present invention, the organism cell auto-handling apparatus comprises a platform, an organelle observation microscope unit, a piping device, and a system controller.

According to another aspect of the present invention, the organism cell auto-handling method is performed through the organism cell auto-handling apparatus, subject to the steps of putting an organism cell sample on the platform, using the organelle observation microscope unit to search the coordinate position of a specific organelle of the organism cell sample and to produce a corresponding coordinate data to the system controller, processing a draw/injection action to the specific organelle through a micro needle of the piping device.

### BRIEF DESCRIPTION OF THE DRAWINGS

FIG. 1 is a schematic drawing of an organism cell auto-handling apparatus according to the present invention.

FIG. 2 is a flow chart showing the operation of the organism cell auto-handling apparatus according to the present invention.

FIG. 3 is a flow chart showing the operation of an alternate form of the organism cell auto-handling apparatus according to the present invention.

### DETAILED DESCRIPTION OF THE INVENTION

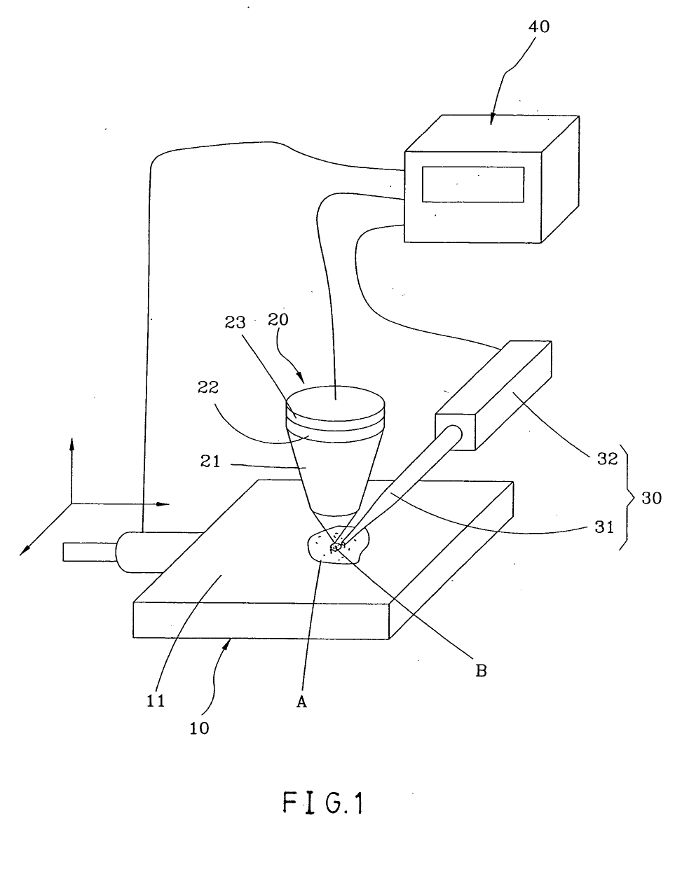

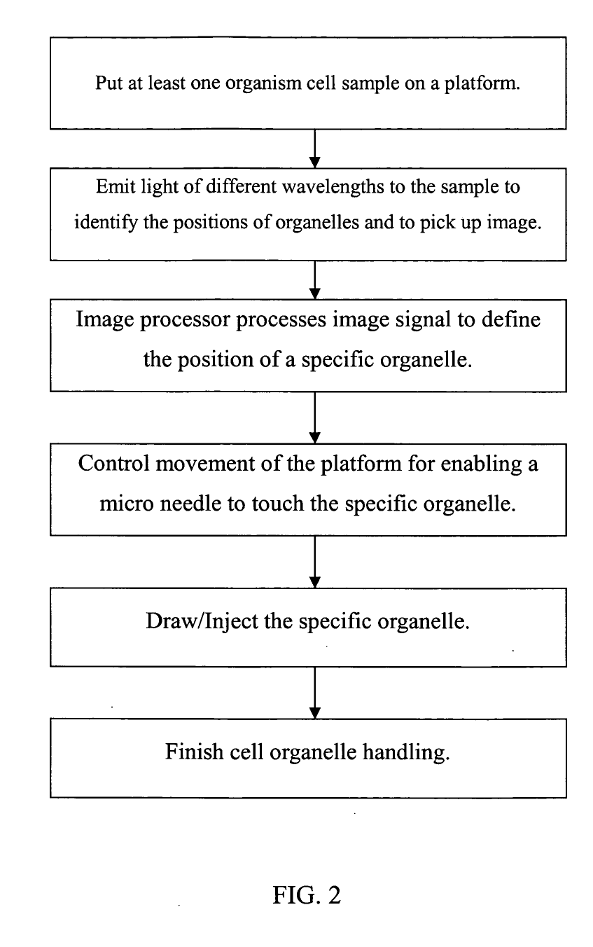

Referring to FIGS. 1 and 2, an organism cell auto-handling apparatus is shown comprising a feed platform 10, an organelle observation microscope unit 20, a piping device 30, and a system controller 40.

The feed platform 10 is electrically connected to the system controller 40 and is precisely movable in X, Y, Z directions by the control of the system controller 40, having a reference surface 11 for holding an organism cell sample A to be examined.

The organelle observation microscope unit 20 comprises a high-power light microscope 21, a programmable multi-wavelength light source (not shown), an image sensor 22 provided at the back side of the high-power light microscope 21, and an image processor 23. When in use, the high-power light microscope 21 is operated to pick up the image of the organelles of the organism cell sample. Since various organelles have different spectrum features and the light source is controlled to emit light of different wavelengths to different organelles of the organism cell sample, the position of the organelles can be identified. And then, the image sensor 22 behind the light microscope 21 transmits the image signal of the organelle of the organism cell sample from the high-power light microscope 21 to the image processor 23 for storage and further processing. Thereafter, the image processor 23 multiplies the pixel signal value of same location in different image signals by a number of different values, and then sums up the total value, and then process the total value through an operation. If the processed pixel signal value surpassed a specific value, the location of a specific organelle B of the organism cell sample is defined, and the coordinate data of the location of such specific organelle B is determined and transmitted to the system controller 40.

The piping device 30 comprises a micro needle 31 made of glass, silicon, or metal. The caliber of the pointed end of the micro needle 31 is slightly greater than the cell's organelle to be picked up/injected. According to the present preferred embodiment, the caliber of the pointed end of the micro needle 31 is within 0.5 to 8μm. The other end of the micro needle 31 is connected to a micro pump 32, which is electrically connected to the system controller 40, and controlled by the system controller 40 to draw or inject such specific organelle B, achieving the organism cell handling process.

In this preferred embodiment, the feed platform 10 is moved relative to the piping device 30 by the control of the system controller 40 to a position convenient for the micro needle 31 to draw or inject a specific organelle of an organism cell. Alternatively, the system of the present invention can be designed to move a piping device 30 relative to a fixed feed platform 10 by the control of the system controller 40 for enabling the micro needle 31 to accurately draw or inject a specific organelle of an organism cell.

As indicated above, the invention has the following advantages.

1. The apparatus and method provided by the present invention provide an auto organelle observation and identification function, enabling the user to rapidly search the location of the specific organelle to be processed, i.e., the function of the present invention effectively accelerates the operation speed.

2. After positioning of a specific cell organelle, the organism cell auto-handling apparatus of the present invention can be precisely positioned to draw the cell organelle accurately. Therefore, the invention improves the operation performance and increases the change of success.

3. The organelle observation microscope unit 20 uses different wavelengths to search the internal organelles of the organism cell sample; such searching does not cause the organelles of the organism cell sample to change their characteristics.

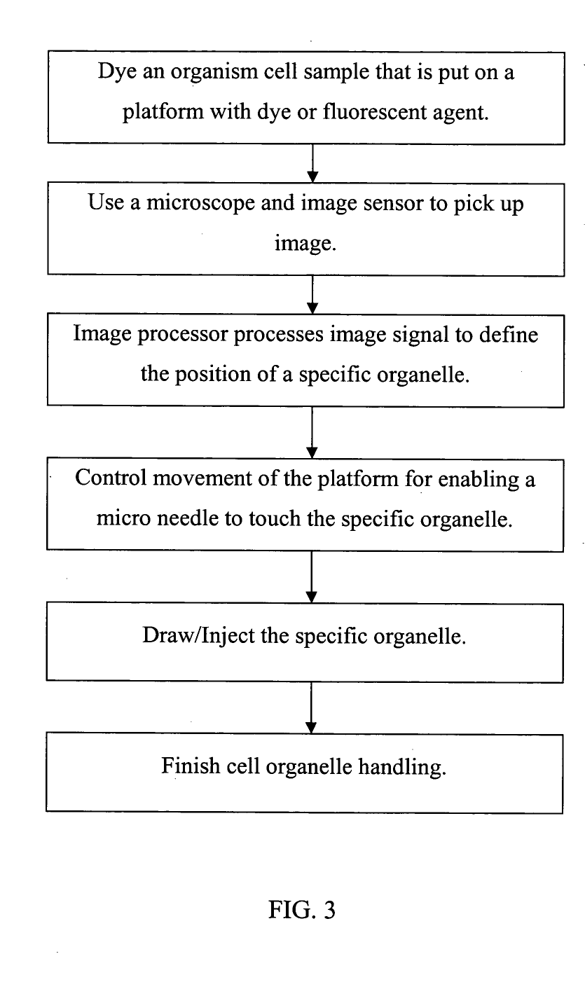

As an alternate form of the present invention, the organelle observation microscope unit of the organism cell auto-handling apparatus is comprised of a high-power light microscope, an image sensor, and an image processor. FIG. 3 shows the operation flow of the organism cell auto-handling apparatus according to this alternate form. As illustrated, the organism cell sample is put on the feed platform, then the organism cell sample is dyed with a dye or fluorescent agent, and then the high-power light microscope and the image sensor are operated to pick up image signals from the organism cell sample. The posterior specific cell organelle positioning and drawing/injecting procedures are same as the aforesaid procedures shown in FIG. 2.

Further, the organelle observation microscope unit can be a confocal microscope unit that directly converts cell organelle image into signal and then transmits the signal to the system controller.

Although particular embodiments of the invention have been described in detail for purposes of illustrated, various modifications and enhancements may be made without departing from the spirit and scope of the invention. Accordingly, the invention is not to be limited except as by the appended claims.

What is claimed is:

1. An organism cell auto-handling apparatus comprising:
a platform for holding thereon at least one organism cell sample;
an organelle observation microscope unit for identifying coordinates of a specific organelle of the organism cell sample for producing a corresponding coordinate data according to the coordinates of the specific organelle;
a piping device having a micro needle for drawing/injecting said organelle of the organism cell sample; and
a system controller for receiving the coordinate data from said organelle observation microscope unit for controlling relative movement between said platform and said piping device according to the received coordinate data for enabling said micro needle to touch the specific organelle of the organism cell sample.

2. The organism cell auto-handling apparatus as claimed in claim 1, wherein said piping device means is fixed in position, and said platform is controllable by said system controller to make at least 2-dimensional motion.

3. The organism cell auto-handling apparatus as claimed in claim 1, wherein said platform is fixed in position, and said piping device is controllable by said system controller to make at least a 2-dimensional motion.

4. The organism cell auto-handling apparatus as claimed in claim 1, wherein said organelle observation microscope unit comprises a microscope, a programmable multi-wavelength light source, and an image sensor.

5. The organism cell auto-handling apparatus as claimed in claim 1, wherein said organelle observation microscope unit comprises a microscope and an image sensor.

6. The organism cell auto-handling apparatus as claimed in claim 1, wherein said organelle observation microscope unit is a confocal microscope.

7. The organism cell auto-handling apparatus as claimed in claim 1, wherein said piping device further comprises a micro pump connected to one end of said micro needle.

8. The organism cell auto-handling apparatus as claimed in claim 1, wherein said micro needle has a pointed end, said pointed end having a caliber greater than the cell organelle to be drawn/injected.

9. An organism cell auto-handling method comprising the steps of:
(a) putting at least one organism cell sample on a platform;
(b) using an organelle observation microscope unit to position a specific organelle of said organism cell sample and produce a corresponding coordinate data to a system controller; and
(c) touching the specific organelle with a micro needle of a piping device that is controlled by the system controller according to the corresponding coordinate data for enabling said piping device to process an organelle draw/injection action.
